(19) United States
(12) Patent Application Publication
(10) Pub. No.: US 2025/0114276 A1
(43) Pub. Date: Apr. 10, 2025

(54) RECONSTITUTION VIAL MIXING APPARATUS

(71) Applicant: Summit International Medical Technologies, Franklin, MA (US)

(72) Inventor: Michael A. Merchant, Franklin, MA (US)

(21) Appl. No.: 18/800,025

(22) Filed: Aug. 10, 2024

Related U.S. Application Data
(63) Continuation-in-part of application No. 17/986,206, filed on Nov. 14, 2022.
Application No. 17/986,206 is a continuation of application No. 16/575,442, filed on Sep. 19, 2019, now Pat. No. 11,497,684.
(60) Provisional application No. 62/879,455, filed on Jul. 27, 2019.
(60) Provisional application No. 62/834,915, filed on Apr. 16, 2019.

(51) Int. Cl. A61J 1/20

(52) U.S. Cl. CPC A61J 1/2089; A61J 1/201; A61J 1/2058

(57) ABSTRACT

A medicinal vial reconstitution and IV dispensing device includes a bifurcated receptacle adapted to engage a medicinal vial, and a valve or barrier for selectively opening and closing the medicinal vial from fluidic communication with the patient IV line. The reconstitution device mixes medication with saline solution from an IV bag through a dedicated channel. The reconstitution device receives a medication vial containing the powdered medication. The reconstituted medication is then passed back into the IV bag for uniform mixing. A valving element ensures fluid flow only from the vial to the bag. Once mixed, the reconstituted medication achieves a desired concentration in the IV bag. The (now medicated) saline solution in the IV bag is dispensed back through the reconstitution device via an IV line to the patient, and the flow control valving element redirects fluid only to the IV line and prevents any backflow with the vial.

### RELATED APPLICATIONS

This application is a Continuation in Part (CIP) under 35 U.S.C. § 120 of earlier filed U.S. patent application Ser. No. 17/986,206, filed Nov. 14, 2022, entitled: “RECONSTITUTION VIAL MIXING APPARATUS,” (Attorney Docket No.: SUM19-02CON), which is a Continuation (CON) of earlier filed U.S. patent application Ser. No. 16/575,442 filed Sep. 19, 2019, entitled: “RECONSTITUTION VIAL MIXING APPARATUS,” (Attorney Docket No.: SUM19-02), which claims the benefit under 35 U.S.C. § 119(e) of U.S. Provisional Patent Application Nos. 62/834,915, filed Apr. 16, 2019, entitled “RECONSTITUTION VIAL MIXING APPARATUS,” and 62/879,455, filed Jul. 27, 2019, entitled “DUAL CHANNEL VIAL MIXING APPARATUS,” all incorporated herein by reference in entirety

### BACKGROUND

IV (Intravenous) lines are commonly used for hydration and medication of patients. The IV line establishes a fluid flow to the patient bloodstream for maintaining a bodily fluid balance, and also provides a convenient medium for medicinal transport, since the IV fluid (typically saline solution) defines a regular flow into the bloodstream from a saline solution bag and is preferable to subjecting the patient to an additional needle injection. IV medications may be mixed into the saline solution bag for delivery with the saline solution. Medications are generally delivered in a concentrated liquid or powder form, and are introduced into the saline solution bag through an access port. Powdered medications need to be reconstituted by mixing with liquid prior to being introduced into the IV bag. The quantity of concentrated medication is generally based on an expected quantity of saline solution for providing an appropriate dosage and introduction rate of the IV delivered medication.

### SUMMARY

A medicinal vial reconstitution and IV dispensing device includes a receptacle adapted to engage a medicinal vial, and a valve or barrier for selectively opening and closing the medicinal vial from fluidic communication with the patient IV line. The valve or barrier imposes a bifurcated, dual channel construction in the device that prevents fluidic mixing between the reconstitution and delivery phases. The reconstitution device mixes powdered medication with saline solution from an IV bag. The reconstitution device receives a medication vial containing powdered medication or drugs, and allows controlled dispensing of a small amount of saline solution from an IV bag into the reconstitution vial (vial). The dispensed saline solution is agitated to mix with the powder medication in the vial for reconstituting the medication into a liquid form. The reconstituted medication is then passed back into the source IV bag for uniform mixing. A flow control valving element flow control valving element ensures fluid flow only from the vial to the bag. Once mixed, the reconstituted medication combined with the volume of saline solution in the IV bag achieves a desired concentration. The (now medicated) saline solution in the IV bag is dispensed through the reconstitution device via an IV port connecting to the IV line to the patient, and the flow control valving element flow control valving element redirects fluid only to the IV line and prevents any backflow or fluid communication with the vial.

Configurations herein are based, in part, on the observation that conventional reconstitution devices have a fixed 3-way fluidic communication between the medicinal vial, IV bag, and IV line. Unfortunately, conventional approaches and devices for IV reconstitution can result in incomplete reconstitution and inadequate mixing. Powdered medication can remain unmixed, or partially mixed, in the vial or in a transfer channel leading back into the vial. Incomplete mixing can affect the concentration of medication in the IV flow to the patient. Residual, unmixed, or partially mixed powder remaining in the vial can result in under concentrated medication, or an overly concentrated mix remaining in the transfer channel may release suddenly into the IV stream, causing a “burst” or “bolus” of medication in the IV line and causing overconcentrated delivery. Accordingly, configurations herein substantially overcome the shortcomings of conventional reconstitution devices by providing a valve connection and/or dual transfer channels to the medication vial, providing fluidic separation between the transfer channel and the IV bag/IV line. Distinct phases of reconstitution, mixing and delivery can therefore be established for mitigating residual medication that can result in a non-uniform IV delivery stream.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a prior art reconstitution mixing device;

FIG. 2 show fluidic flow control in the disclosed reconstitution device;

FIG. 3 shows a perspective view of the reconstitution device of FIG. 2;

FIG. 4 a cutaway view of the reconstitution device of FIG. 2 including a flow control valving element;

FIG. 5 shows a cutaway view of the reconstitution device of FIG. 2 including an alternate flow control valving element;

FIG. 6 shows a valve in the device of FIG. 4;

FIG. 7 shows an open valve in the device of FIG. 4;

FIG. 8 shows an axial view of the valve in FIGS. 6 and 7 progressing between open and closed positions;

FIG. 9 shows an alternate valve implementation disposing the valve of FIGS. 6 and 7 at a protrusion in the IV stream for promoting drug mixing;

FIG. 10 shows a combined valve and partition (dual channel) arrangement of the reconstitution device;

FIG. 11A-11B shows respective schematic views of a single and dual channel reconstitution device structure.

FIGS. 12A-12B show a schematic of the valve channel of FIGS. 11A-B;

FIGS. 13A-13B show a cutaway view of the valve having concentric sections adjacent to the receptacle;

FIG. 14 shows a cutaway view of the valve having an elongated concentric section extending from the receptacle;

FIG. 15 shows an axial transparent view of the concentric sections of FIGS. 12A-14;

FIGS. 16A-16C show the valve progression through an open, closed and locked position;

FIGS. 17A-17C show an alternate configuration having a recession and protrusion engagement on the opposed concentric sections for defining valve open/closed positions;

FIGS. 18A-18C show an axial view of the recessions and protrusions on the opposed concentric sections defining the valve and locking positions;

FIGS. 18D-18G show a side cutaway view of the concentric sections of FIGS. 18A-18C

FIGS. 19A-19C show an alternate valve construction in the valve channel; and

FIGS. 20A-20C show and alternate locking configuration for the valve of FIGS. 19A-19C.

### DETAILED DESCRIPTION

The disclosed reconstitution device takes the form of an Intravenous (IV) connector adapted to be disposed in an IV line for delivering saline solution or similar IV fluid containing the reconstituted medication to a patient via an IV line. Reconstitution involves extracting a small portion of the saline solution from the IV bag into the vial, reconstituting the medication in a mixture of the saline solution and concentrated medication, then passing the reconstituted mixture back through the dedicated dual channel IV connector to mix with the full quantity of saline solution in the IV bag. The same IV connector also leads to the patient IV line, and the reconstituted medication flows back out of the bag but passes only to the patient IV line, and does not “backwash” or flow into the vial due to a flow control valving element directing all the medicated IV saline solution only to the IV line.

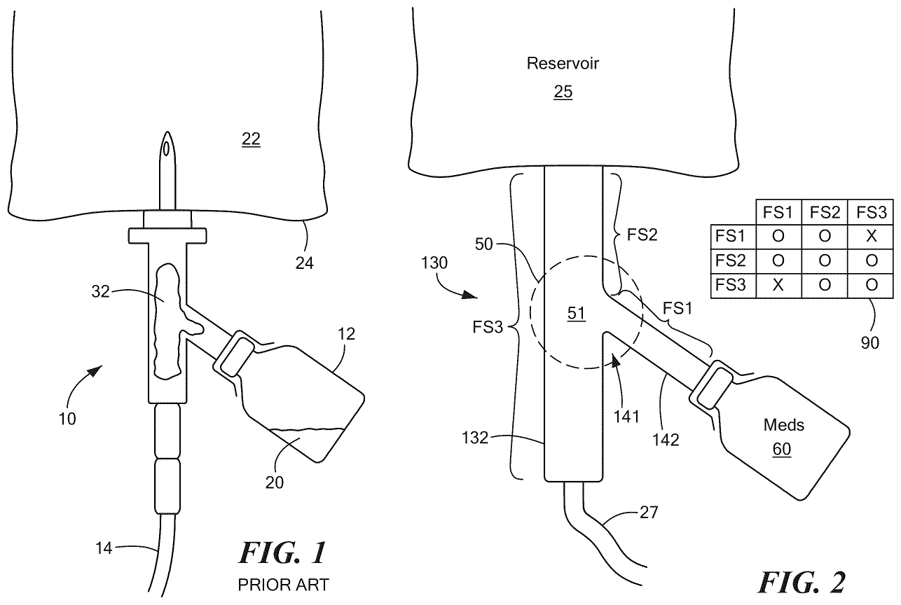

FIG. 1 is a prior art reconstitution mixing device. Referring to FIG. 1, a conventional delivery device 10 allows medication delivery from a conventional medication vial 12 to a patient though a conventional IV line 14. Concentrated medication 20 in the medication vial 12 needs to be reconstituted by mixing with saline solution 22 or other delivery fluid from an IV bag 24. Saline solution 22 from the IV bag 24 is mixed with the medication 20 by forcing the saline solution into the vial 12 through a dedicated flow channel, and returning the concentrated medication to the IV bag 24 as a conventional mixture of saline solution and medication. The quantity of concentrated medication 20 is intended for mixing with an entire volume of saline solution 22 in the IV bag 24 for proper administration.

A mixing region 32 is defined in the conventional delivery device 10 at a fluid junction of the medication vial 12, the IV bag 24, and the IV line 14. Medication is expected to pass through the conventional mixing region at least twice—once as the concentrated medication exits the vial 12 and travels to the IV bag 24, and again as the reconstituted medication (meaning mixed at a correct or expected concentration) passes from the IV bag 24 to the conventional IV line 14. Since the conventional mixing region 32 connects each of the vial 12, IV bag 24, and IV line 14, concentration irregularities can result in the mixing region 32 which cause the conventional IV line 14 to deliver medication at higher or lower concentrations than the expected concentration.

FIG. 2 shows fluidic flow control in the disclosed reconstitution device. In the reconstitution device 100, saline solution used for reconstituting the medication is also used for delivering the medication. A small amount of saline solution is transferred to the vial for reconstitution through a dedicated flow channel, resulting in a concentrated medication mixture or solution in the vial. This concentrated liquid in the vial is then transferred back into the IV bag where it mixes with the remaining saline solution to result in a proper concentration for patient delivery. During the reconstitution step, an indefinite and possibly harmful concentration of powdered or liquid medication exists in the vial. It is beneficial if this intermediate concentration is directed for combination with the IV bag prior to any release into the patient IV line. Otherwise, portions of concentrated and/or unmixed medication may inadvertently remain in the mixing region 32 and pass to the patient IV line prior to complete mixing and dilution with a full volume of saline solution in the IV bag. The flow control imposed by the valve or barrier imposes a divided, dual channel construction in the device that prevents fluidic mixing between the reconstitution and delivery phases.

Accordingly, the flow control valving element in the IV connector defines a plurality of fluidic flow control segments though the IV connector, such that the medicinal fluid traverses the fluidic flow control segments in a predetermined sequence. The predetermined sequence directs the flow of saline solution, reconstituted medication, and properly diluted medication between a medication source 60 such as a vial, a fluid reservoir 25 such as a saline solution bag, and an IV line 27 or IV tubing to a patient. A first flow segment FS1 extends from a transfer channel 142 ingress to the IV connector 130. A second flow segment FS2 extends from the transfer channel 142 into the fluid reservoir 25, and a third flow segment FS3 extends from the fluid reservoir 25 to the patient IV line 27 through the IV connector 130, such that the flow control valving element is disposed for directing fluid from the first flow segment to the second flow segment prior to the fluid traversing the third flow segment. A fluid communication table 90 shows allowed fluid paths, and indicates that medication fluid cannot pass directly from FS1 to FS3, thus forcing all fluid thorough the saline solution bag for proper concentration.

A flow control valving element 50 is disposed at or near the junction 51 of the flow control segments FS1 . . . FS3 for moderating fluid flow though the junction 51 for enforcing proper concentration of delivered medicine. As a result of the flow control valving element 50, the second FS2 and third flow control segments FS3 travel in opposed directions though the IV connector 130. The flow control valving element 50 may be any suitable fluidic barrier suitable for enforcing the flow control segments and fluidic communication between the flow control segments, the receptacle, IV bag and patient IV line 127 via the IV connector. In particular configurations, the flow control valving element may be a valve or partition for directing and/or restricting the flow control segments.

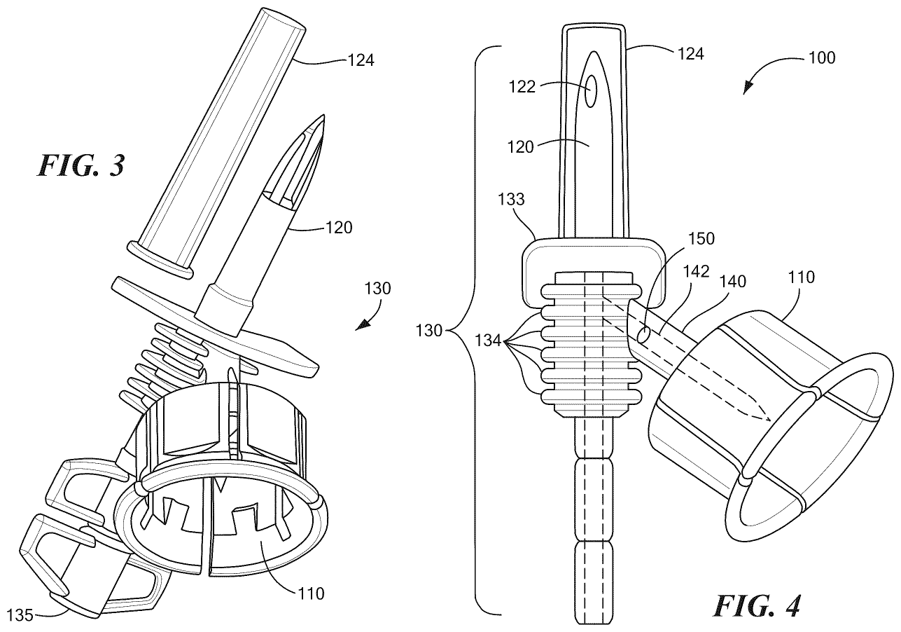

FIG. 3 shows a perspective view of the reconstitution device of FIG. 2. Referring to FIGS. 2 and 3, the IV connector 130 includes an IV spike 120 adapted for insertion into an IV bag defining the reservoir 25, a cap 124, a receptacle 110 for engaging a medicinal vial containing the medication 60, and an IV outlet 135 including one or more IV tabs 137 for engagement by the patient IV line 27. In operation, the IV connector 130 facilitates IV delivery of a vial based medication concentration requiring reconstitution.

FIG. 4 a cutaway view of the reconstitution device of FIG. 2 including the flow control valving element 50. Referring to FIGS. 2-4, the medicinal vial reconstitution and dispensing apparatus 100 includes a receptacle 110 adapted to engage a medicinal vial, and an intravenous (IV) connector 130 adapted for inline connection to a patient IV line. The IV connector 130 is adapted for transport of medicinal fluids to a patient via an aperture 122 or passage in a tapered end defining an IV spike 120, and remains covered by the cap 124 until just prior to use. A bifurcated junction body 140 extends from a longitudinal side of the IV connector to the receptacle 110, and has a transfer channel 142 adapted for fluidic communication between the receptacle 110 and the IV connector 130 for transferring liquids between the vial and IV connector 130. The flow control valving element 50 defines a selective closure such as a valve 150 or other fluid retention and/or interference mechanism in the bifurcated junction body 140 that allows selectively opening and closing of the transfer channel 142 for sealing the medicinal vial from an IV channel 132 through the IV connector 130. Ribs 134 around the junction 51 of the transfer channel 142 and IV channel 132 provide reinforcement for the transfer channel valve 150, discussed further below. A flange 133 region extending outward from the connector 130 may have a textured or dimpled surface to facilitate a frictional grip during IV bag insertion.

In FIG. 4, the bifurcated junction body 140 extends from the IV connector 130 to the receptacle 110, and encapsulates the transfer channel 142 for fluidic communication between the receptacle 110 and the IV connector 130 for transferring liquids between the vial and IV connector 130. The flow control valving element 50 enforces a controlled flow path through the IV connector 130, such that the controlled flow path directs fluid from the transfer channel 142 to the fluid reservoir 25 before passing to the patient IV 27 line through the IV connector. The transfer channel valve 150 disposed along the transfer channel 142 defines the flow control valving element 50. In this arrangement, the flow control valving element 50 selectively impedes fluidic flow through the transfer channel 142 for preventing unwanted flow to the patient IV line 27, discussed further below. Therefore, the second flow segment FS2 and the third flow segment FS3 pass thorough the IV connector 130 and into and out of the reservoir 25. The flow control valving element 50 therefore directs a fluidic path from the vial through the reservoir 25 prior to fluidic flow into the patient IV line 27.

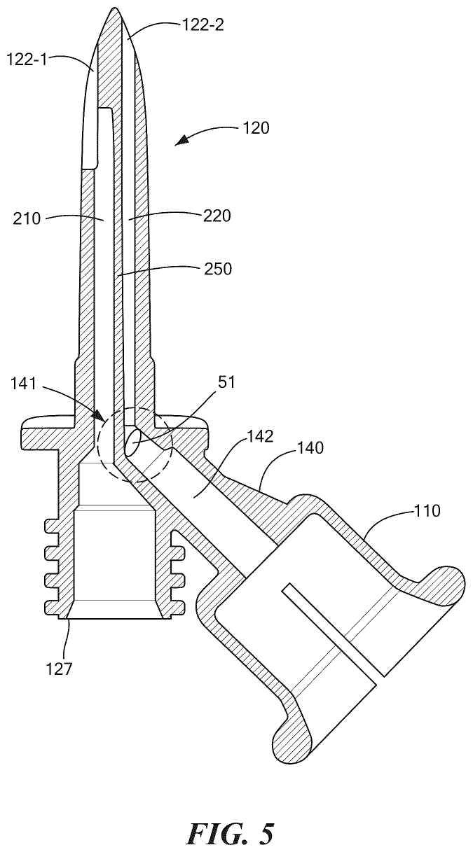

FIG. 5 shows a cutaway view of the reconstitution device of FIG. 2 including an alternate flow control valving element 50. Referring to FIGS. 2 and 5, the flow control valving element 50 includes a partition 250 that defines a dual channel approach in the device of FIG. 2 for managing the flow control segments FS1 . . . FS3. Referring to FIGS. 2-5, a first channel 210 extends from the bag spike 120 to the patient IV line 27 via an IV port 127, and defines flow segment FS3. A second channel 220 extends from the bag spike 120 to the junction 51, defining flow segment FS2. As a result of the partition 250 extending to the junction 51, the second channel 220 connects only to the transfer channel 142, and does not permit fluidic communication with the first channel 210 or the patient IV line 27, thus defining flow segment FS1 from the receptacle 110.

The bifurcated junction body 140 encloses the transfer channel 142 defining the first flow segment FS1, connecting with the second channel 220 to define flow segment FS2 at bifurcation 141. The IV connector 130 includes the IV bag spike 120 adapted for insertion into an IV bag 125 defining the fluid reservoir 25. The second channel 220 is therefore adapted for fluidic communication between the receptacle 110 and the IV bag 125 via the IV bag spike 120. The first channel 210 is adapted for transferring liquids between the IV bag 125 and IV port 27 via the IV bag spike 120 when inserted into the IV bag.

It should be noted that the device exhibits outlet ports, or openings, at an emergence of each of the flow control segments for fluid flow to one of a medicinal source, typically a vial, a repository, typically an IV bag, and an IV connector, typically to a patient delivery IV line. The device maintains the flow control segments FS1-FS3 for controlling and directing medicinal fluid through interior channels in the device. Each such channel is independent of a port or opening through which it may deliver or receive fluid. In other words, the device need not engage the bag spike by a single channel, but rather the bag spike may have two channels with respective orifices in fluidic communication with a fluid volume in the bag.

Based on the partition 250, the IV connector 130 has separate, dual channels 210, 220 defined by the flow control valving element 50, such that each channel corresponds to one of the second or third flow control segments. In this arrangement, the flow control valving element 50 divides fluid flow between an ingress channel and an egress channel to the fluid reservoir 25. The flow control valving element 50 defines a first channel 210 and a second channel 220 through the IV connector 130. The first channel 210 defines fluidic communication between the fluid reservoir 25 and the patient IV port 127/line 27, and the second channel 220 defines fluidic communication between the receptacle 110 and the fluid reservoir 25. The IV bag spike 120 further includes a first orifice 122-1 at an end of the first channel 210 and a second orifice 122-2 at an end of the second channel 220, such that each of the first orifice and the second orifice are in fluidic communication with the fluid reservoir 25 for fluidic transfer between the respective channels. In terms of the flow control segments of FIG. 2, the second FS2 and third FS3 flow control segments travel though separate ingress and egress orifices in the bag spike 120 between the IV connector 130 and the reservoir 25.

A vial 60 engaged in the receptacle 110 connects to the transfer channel 142 that leads to the second channel 220 and then into the bag spike 120 and attached IV bag or reservoir 25 for exchanging reconstitution fluid (saline solution) with the bag. The mixed, reconstituted medicine that has already been exclusively forced into the bag may now exit the bag and into the IV line 27 via the first channel 210. In operation, the flow control valving element 50 defines an internal fluidic path for transporting fluid from the vial to the patient IV line 27 that completely traverses the second channel 220 followed by the first channel 210. In this manner, the flow control valving element 50 imposes a fluidic barrier between the first channel 210 and the receptacle 110.

The external bifurcated appearance of an IV line connector between the IV bag and a downstream IV port with an angled branch for the vial should be distinguished with respect to the channels through the device. Controlled fluid flow is effected by the internal channels, which connect the outlets or ports as defined herein. The internal channels are the passages through the device in which fluid flows. Channels, therefore, define fluidic communication between the three endpoints of the device, and not the exterior outlets or ports which receive and/or dispense fluid.

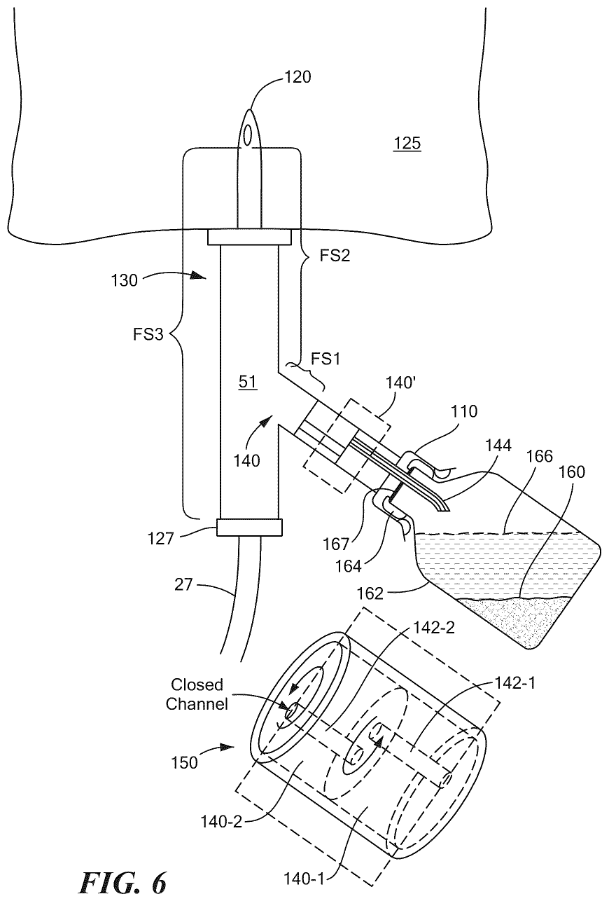

FIG. 6 shows a valve in the device of FIG. 1. During administration of a reconstituted drug, the powdered medication 160 arrives in a vial 162. The receptacle 110 has a vial holder and a spike 144 adapted for penetration of a resilient portion in a cap on the vial 162. The vial 162 has a collar 164 adapted for engagement by the receptacle 110, usually by a frictional and/or interference fit from subtle deformation of the resilient tabs on the receptacle 110. The vial holder deformably slides onto the vial top and resiliently “snaps” into place, where it attains an appearance of an apron around the vial cap. A puncture region 167 is an elastomeric or membrane material adapted for a sealing engagement with a cannulated spike 144 or needle that pierces the puncture region 167 for establishing fluid communication with the transfer channel 142 in the bifurcated junction body 140 that includes FS1 and FS2. Alternatively, a needleless or luer connector may connect to the vial 162.

Substantially concurrently, the IV connector 120 attaches to a saline solution bag 125. The IV connector 120 invokes the tapered end adapted for engaging a suitable IV saline solution bag 125. The fluid contents of the saline solution bag 125 provide both reconstitution fluid for the medication 160 and the IV fluid medium for patient transport. After attaching the saline solution bag 125, the connector 130 is inverted and the IV bag compressed to force saline solution into the vial 162 via FS1 and FS2. After a sufficient fluid level 166 is introduced into the vial 162, an assistant (nurse, technician) agitates the vial to reconstitute (mix) the saline solution and medication 160 into a solution or suspension. The connector 130/saline solution bag 125 combination is oriented with the vial 162 upwards so that the trapped air displaces up into the vial 162 and forces the reconstituted medicine down into the saline solution bag 125, again through FS2 and FS1, ideally defining a known medicinal concentration in the IV bag 125.

Conventional approaches leave this vial/bag combination in place for simply permitting the contents of the bag 125 to flow via a patient IV line 27 at an opposed end of the connector from the IV bag, as is common with IV delivery. Configurations herein dispose a valve 150 in the transfer channel 142 to define a fluidic separation from the vial 162 during the post-reconstitution medicinal delivery phase. This avoids subsequent introduction of any residual medication that may remain in the transfer channel 142 that could alter the concentration of the IV medication delivered to the patient.

In particular configurations, the valve 150 is to shut off the flow channel at the juncture of the transfer channel 142 and the IV flow channel 132 so that there is a reduced possibility of residual fluid in the transfer channel 142. The valve 150, therefore, may be located at the top of the fluid channel from the vial to the spike 144, as near the vial 162 as practicable. Alternatively, a valve 150 may be placed at the juncture of the transfer channel 142 and IV channel 132, discussed below, or at an intermediate position along the transfer channel 142 for facilitating manufacturing. Medication from the IV bag is then delivered via FS3, and cannot traverse back through FS1.

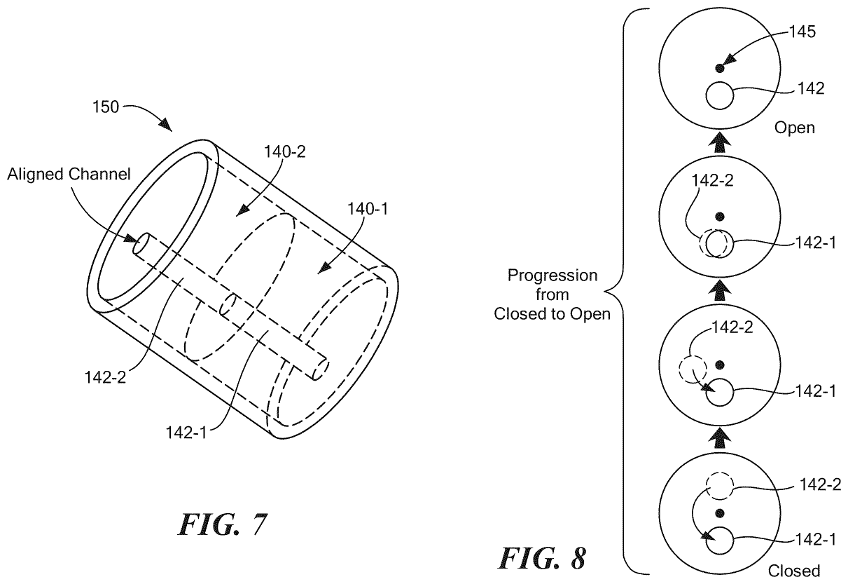

FIG. 7 shows an open valve in the device of FIG. 6. The valve 150 may take the form of an off-center divided channel adapted for rotation in and out of alignment, shown exploded as dotted line segment 140′. Referring to FIGS. 6 and 7, the transfer channel 142 is defined by a vial side portion 142-1 and an IV side portion 142-2. The valve 150 further comprises concentric sections 140-1, 140-2 (140-N generally) of the bifurcated junction body 140 adapted to rotate individually for aligning the transfer channel 142 for fluid communication between the vial 162 and IV channel 132. The transfer channel 142 extends through the plurality of concentric sections 140 in the body, in which the concentric sections 140-N define a selectively opening passage depending on alignment of the channel portions 142-1 and 142-2. FIG. 7 shows an open valve in the device of FIG. 6 as the concentric sections 140 rotate to align the channel portions 142 for permitting saline solution mixing, reconstitution and reconstituted medicinal flow back into the IV saline solution bag 125.

FIG. 8 shows an axial view of the valve in FIGS. 6 and 7 progressing between open and closed positions. The transfer channel 142 extends through a plurality of concentric sections 140 in the body, such that the concentric sections 140 define the valve and are adapted to rotate for aligning and unaligning the transfer channel 142. Unaligned concentric sections 140 act as a barrier and result in a discontinuous transfer channel as each portion 142 is blocked by the unaligned adjacent section 140-N.

The concentric sections 140 define the valve and are adapted to rotate for aligning and unaligning the transfer channel 142 as one concentric section 140-1 rotates relative to the other 140-2, and the transfer channel portion 142-2 is drawn out of alignment with the adjacent transfer channel portion based on the offset of the transfer channel portions from a rotational axis 145. The transfer channel 142 is therefore defined by the portions 142-1, 142-2 in each of the concentric sections 140, such that the portions are offset from a rotational axis 145 of the concentric sections for aligning the portions based on rotation of the concentric sections 140.

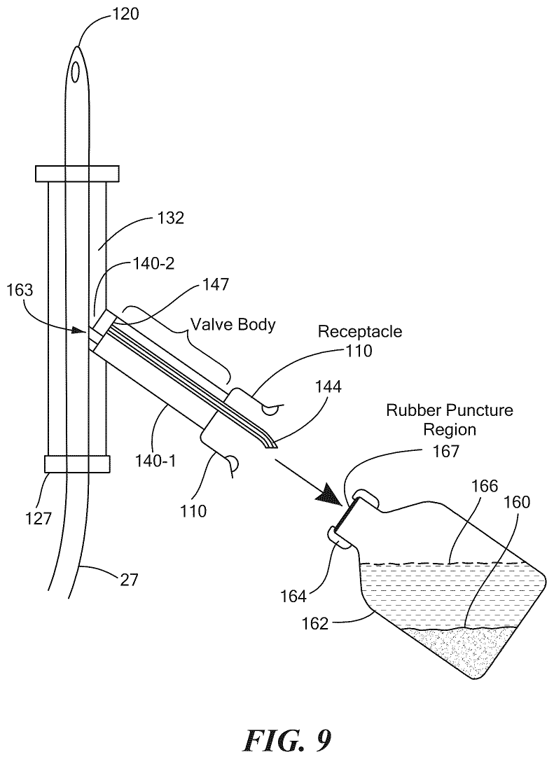

FIG. 9 shows an alternate valve implementation disposing the valve of FIGS. 6 and 7 at a protrusion in the IV stream for promoting drug mixing. FIG. 5 shows an alternate valve implementation disposing the valve of FIGS. 6 and 7 at a protrusion 163 in the IV stream for promoting drug mixing. In FIG. 9, a separation 147 between the IV side portion 140-2 and vial side portion 140-1 is disposed closer to the IV channel 132 such that it occupies a protrusion 163 in the IV channel 132. This arrangement reduces the length and volume of the channel portion 142-2 remaining in fluidic communication with the IV channel 132, minimizing a volume which could contain unmixed medication 160. Also, the protrusion 163 agitates the IV fluid flow to prevent any kind of vortex effect or current which could cause residual droplets or quantities of concentrated, reconstituted medication to remain. The construction of the juncture between the transfer channel 132 and IV channel 142 may therefore either be flush or exhibit the protrusion 163, depending on the location and construction of the valve 150.

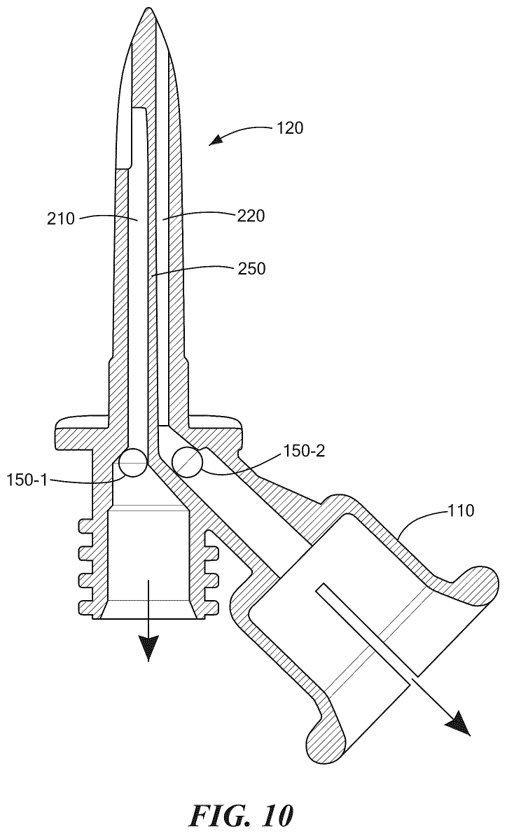

FIG. 10 shows a combined valve and partition arrangement of the reconstitution device. Referring to FIGS. 2-5 and 10, the medicinal vial reconstitution and dispensing apparatus includes an IV bag spike 120 and the receptacle 110 adapted to engage a medicinal vial from the second channel extending through the IV bag spike 120. An intravenous (IV) connector 130 is adapted for inline connection to the patient IV line or IV set, such the IV connector is adapted for transport of medicinal fluids to a patient from the first channel 210 extending through the IV bag spike 120. The bifurcated junction body or ribbed housing extends from the bag spike to the IV connector and joins the receptacle for enclosing the first channel 210 and the second channel 220. The second channel 220 is adapted for fluidic communication between the receptacle 110 and the IV bag via the IV bag spike and the first channel 210 is configured for transferring liquids between the vial and IV connector.

One or more valves 150-1 . . . 150-2 in the connector 130 and/or bifurcated junction body 140 allow selectively opening and closing at least one of the first channel 210 and the second channel 220. As in the configuration of FIG. 4, a valve 150-2 on the second channel 220 is for sealing the medicinal vial 162. Alternatively or in addition, a valve 150-1 on the first channel 210 can seal the IV line from the bag, possible to ensure more complete mixing of the reconstituted medicine before patient delivery.

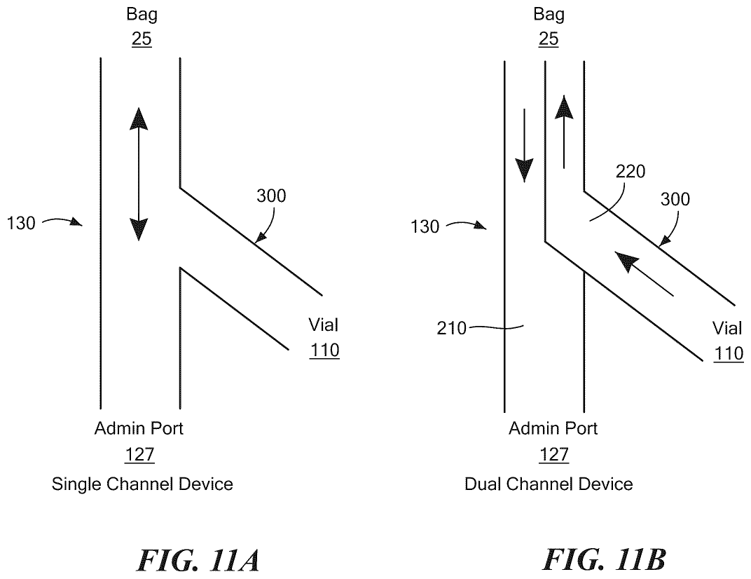

In an alternate configuration, the IV connector 130 employs a valve channel 300 extending from the IV connector 130 (connector) to the receptacle 110 for fluidic communication with the spike 144 and vial 162. Continuing to refer to FIGS. 4-6, a valve channel 300 employs a valve for control of fluid flow to and from the receptacle 110 via the spike 144. FIG. 11A-11B show respective schematic views of a single and dual channel reconstitution device structure adapted for employing a valve. Referring to FIGS. 11A-11B, and continuing to refer to FIGS. 4-6, a general arrangement for the valve channel 300 is shown. The connector body 130 may take either a single channel (FIG. 11A) or dual channel (FIG. 11B) design. While both are effective, the dual channel approach separates the first channel 210 from the fluid reservoir 25 to the IV port 127, and a second channel 220 from the spike 144 to the reservoir 25. The valve channel 300 directs fluid to the fluid reservoir 25 before passing to the patient IV line through the IV connector 130 at an IV output 127. This allows a dedicated path for reconstituted medication to flow to the reservoir 25 for achieving a proper concentration based on the fluid (saline) volume in the bag. This also avoids a fluidic path from the spike 144 to the IV port 127, which bypasses the reservoir 25 and can cause improper concentrations of medication to reach the patient. Industry trends have progressed towards favoring a dual channel approach for added safety.

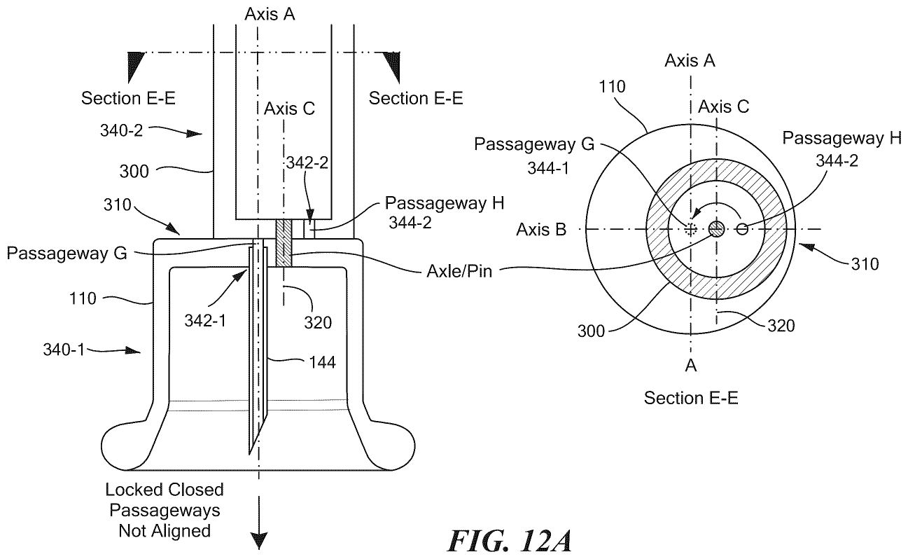

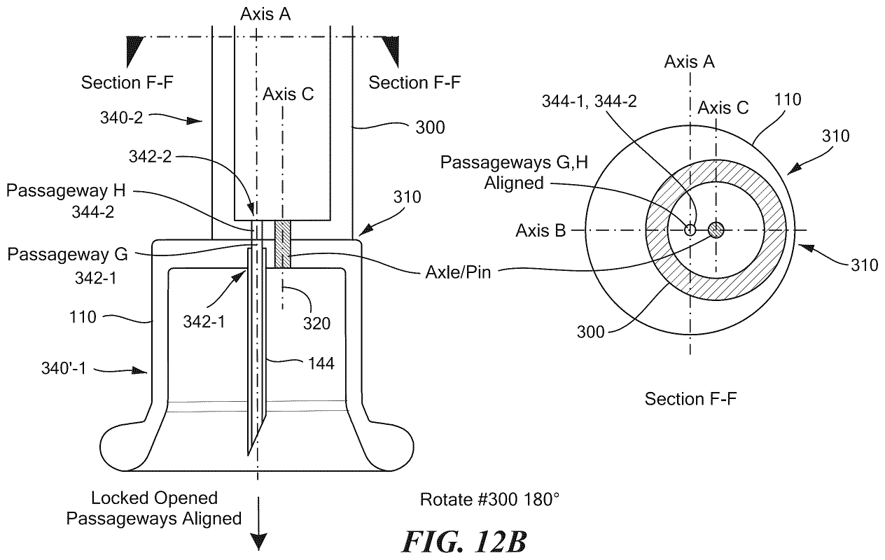

FIGS. 12A-12B show a schematic of the valve channel 300 of FIGS. 11A-B. Referring to FIGS. 11A-12B, the valve channel 300 includes a rotational engagement with the receptacle 110 for forming a valve 310 for interrupting fluid passage through the transfer channel 142. In the configuration of FIGS. 11A-12B, the valve channel 300 forms a bifurcated junction body extending from the IV connector 130 to the receptacle 110. The valve channel 300 positions the valve 310 for selectively opening and closing the valve channel 300 for sealing the medicinal vial 162 and any contents therein. The valve channel 300 is therefore adapted for fluidic communication between the receptacle 110 (via the spike 144) and the IV connector 130 for transferring liquids between the vial 162 and IV connector 130. In the example arrangement, the valve channel 300 is adapted to receive the receptacle 110 in a rotational engagement for selective alignment with an off-center bore.

The disclosed approach shows a valving mechanism that provides flow control. In some configurations, a plurality of concentric sections adapted to rotate individually for aligning the valve channel for fluid communication. Any suitable fluidic interference may be employed, however configurations herein demonstrate a first concentric section 340-1 in rotational communication with a second concentric section 340-2 (340 generally). Each concentric section 340 includes a respective channel portion 342-1, 342-2 (342 generally), respectively, each having an offset passage 344-1, 344-2, (shown as passageways G and H respectively) defined by a bore or orifice defining the respective channel portion 342. When respective channel portions 342 align, as a result of the offset passages 344 being moved so as to oppose each other, an open region or vessel is defined for permitting fluid flow.

Rotational communication, therefore, brings the offset passages 344-1, 344-2 (344 generally) into alignment for allowing fluid flow in an open position and restricting or closing fluid flow in a closed, unaligned position. The positioning of the offset passages 344 distal from an axis 320 of rotation of the respective channel portions 342 allows relative rotation of the concentric sections 340 to bring the offset passages 344 in and out of alignment for attaining an open position 340′-1 of the receptacle 110. “Concentric” in this context refers to the rotation of the concentric sections 340 around a common axis 320, although the geometric “centers” of each portion are slightly offset to effect closure and valve 310 operation, as described below.

The concentric sections 340 may reside at any position along a length of the valve channel 300, for accommodating varying lengths and angles of the channel portions 342 for defining the alignment of the offset passages 344. It can be observed that valve channel 300 extends through a plurality of concentric sections 340 (typically 2) in the body, such that the concentric sections 340 define the valve 310 and are adapted to rotate for aligning and dis-aligning the offset passages 344. In alternate configurations, the concentric sections 340 may be angular offset, and moving about an off-center axis in a scissors-like movement, and may provide for opening, closing, and locking of the concentric sections 340 for blocking unintended medicinal flow. Regardless of the rotation, the channel portions 342 offset from a rotational axis 320 of the concentric sections for aligning the portions based on rotation of the concentric sections 340 to allow communication between the offset passages 344.

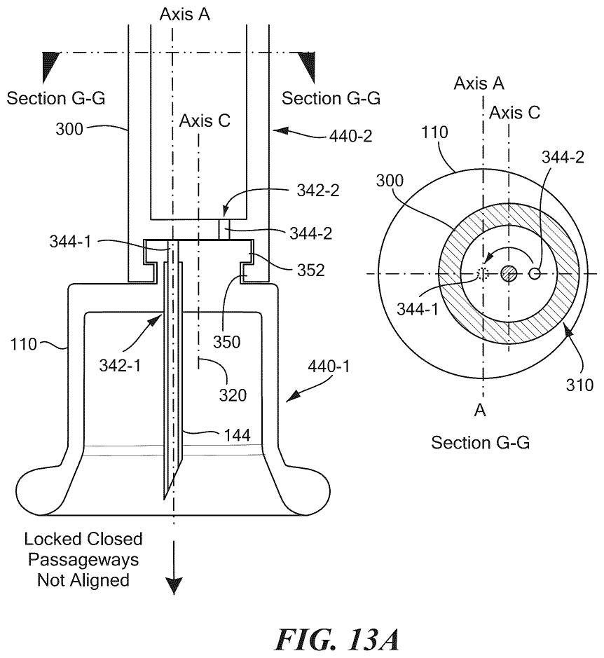

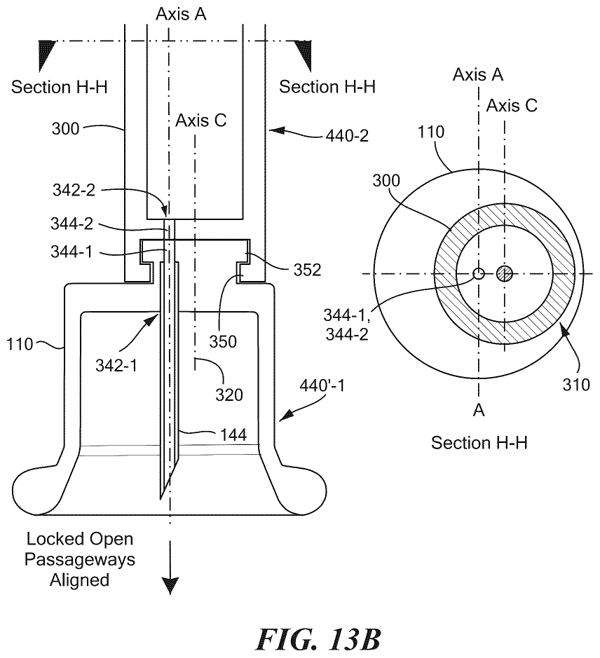

FIGS. 13A-13B show a cutaway view of the valve having concentric sections 440-1, 440-2 (440 generally) adjacent to the receptacle 110. The valve channel 300 may dispose the concentric sections 440 in any suitable arrangement for implementing the valve 310. Referring to FIGS. 13A-13B, a molded ring 352 has a larger diameter for forming an interference fit with a protrusion 350 at a distal end of the transfer channel 300. The molded ring 352 forms a continuous piece with the receptacle 110 and a rotational collar connects the receptacle 110 with the molded ring 352. The molded ring 352 defines the first channel portion 342-1, rotated by rotating the receptacle 110. The offset passage 344-1 aligns with the offset passage 344-2 on the 2^{nd} concentric section 440-2 when the receptacle is rotated, progressing to an alignment of the offset passages 344, shown in FIG. 13B, and opening the valve 310.

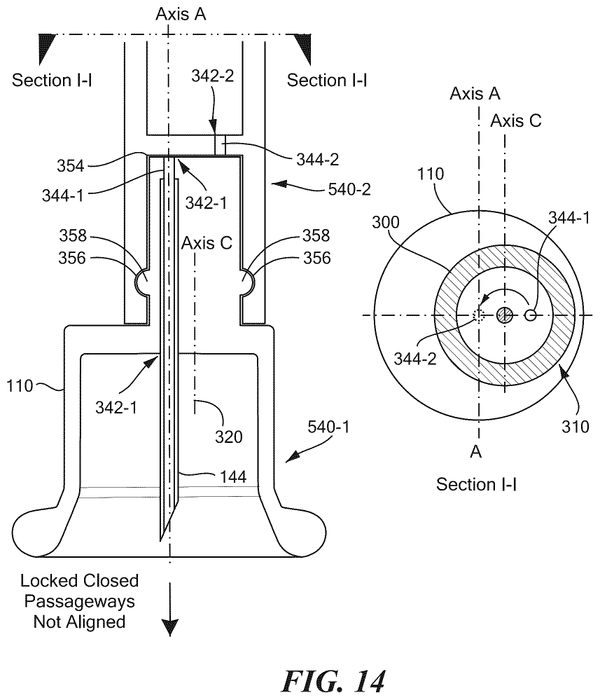

FIG. 14 shows an alternate configuration with the valve 310 having an elongated concentric section 540-1 extending from the receptacle 110 and having a corresponding channel portion 342-1. The valve channel 300 has a corresponding opening bore 354 in the concentric section 540-2 for receiving the concentric section 540-1, and forms a circular groove 356 for engaging a ridge 358 on the channel portion. Other suitable arrangements include some form of a flange and rib formed with the IV connector at a junction of the IV connector and the valve channel, or at any position along the valve channel 300.

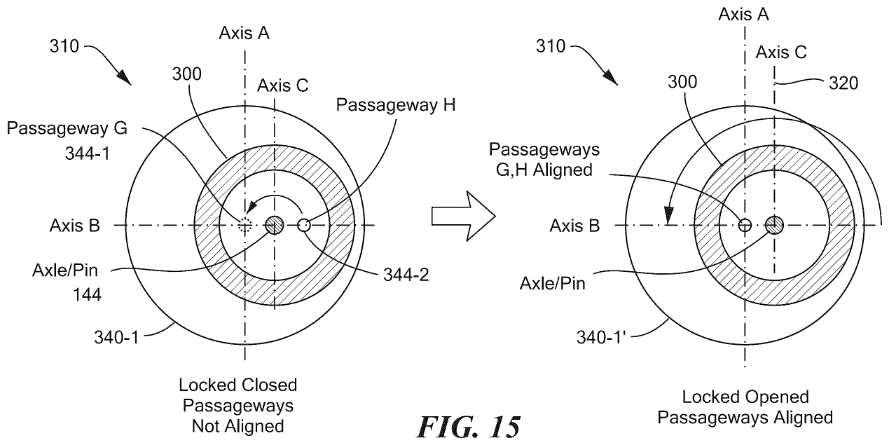

FIG. 15 shows an axial transparent view of the transition of the concentric sections of FIGS. 12A-14 corresponding to opening and closing of the valve 310. Referring to FIG. 15, in a closed position the first concentric section 340-1 has a first offset passage 344-1 shifted from the centered spike 144 so that rotation alters the relative position to the second offset passage 344-2. When rotated, the second offset passage 344-2 is aligned with the first offset passage 344-1 to align the offset passages 344 (and corresponding channel portions 342) in a valve 310 open position.

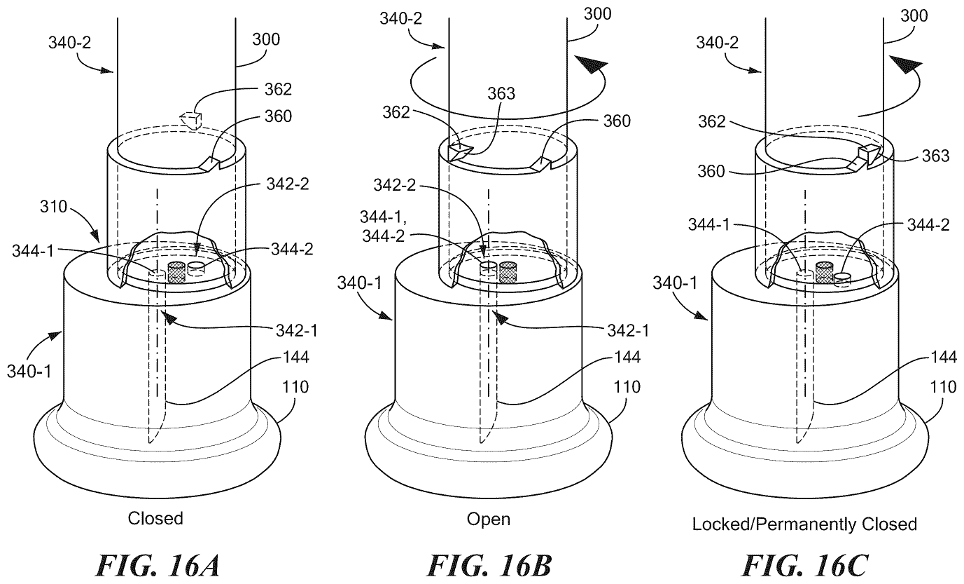

FIGS. 16A-16C show the valve progression through an open, closed and locked position. Referring to FIGS. 16A-16C, in an alternate arrangement, a third valve position achieves a locked state such that the fluidic communication to the vial is permanently terminated. The locked state is beneficial in single use contexts and to avoid duplicate dosages by alerting caretakers of previous use and medicinal administration through the device. In FIG. 16A, the offset passages 344 are out of alignment and the valve is closed. In this configuration, a locking tab 360 extends from the first concentric portion 340-1, and the second concentric portion 340-1 has a mating protrusion (protrusion) 362. FIG. 16B shows rotation of the first concentric portion 340-1 to align the offset passages 344 and open the valve 310 as the locking tab 360 moves towards the protrusion 362. At FIG. 16C, the first concentric portion 340-1 rotates to a locked position as a beveled surface 363 of the locking tab 362 slides across the protrusion 362 and slightly deforms to “snap” into a locked position as the locking tab 360 passes the protrusion 362, leaving the offset passages 344 out of alignment in a locked and closed position. Such a position is helpful following reconstitution to ensure that reservoir 25 contents do not flow back into the vial and also to indicate that the vial 162 has been administered.

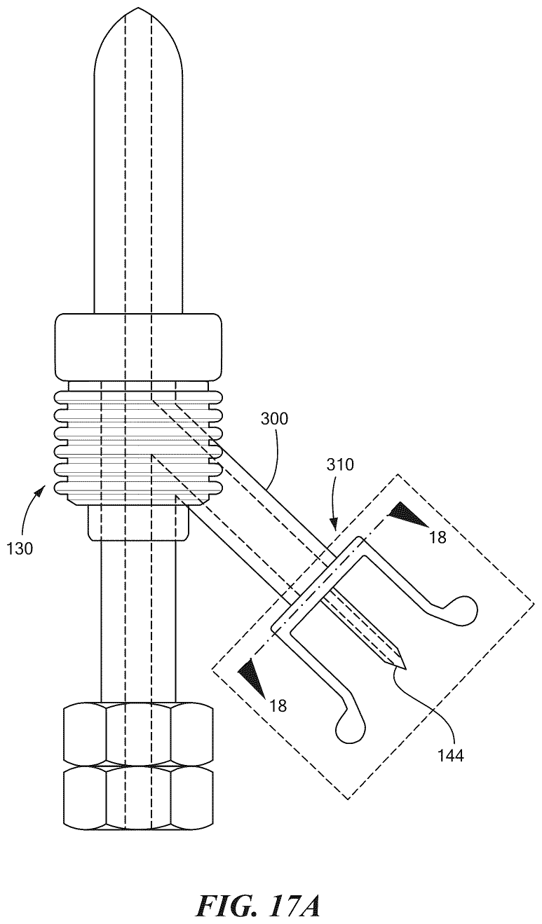

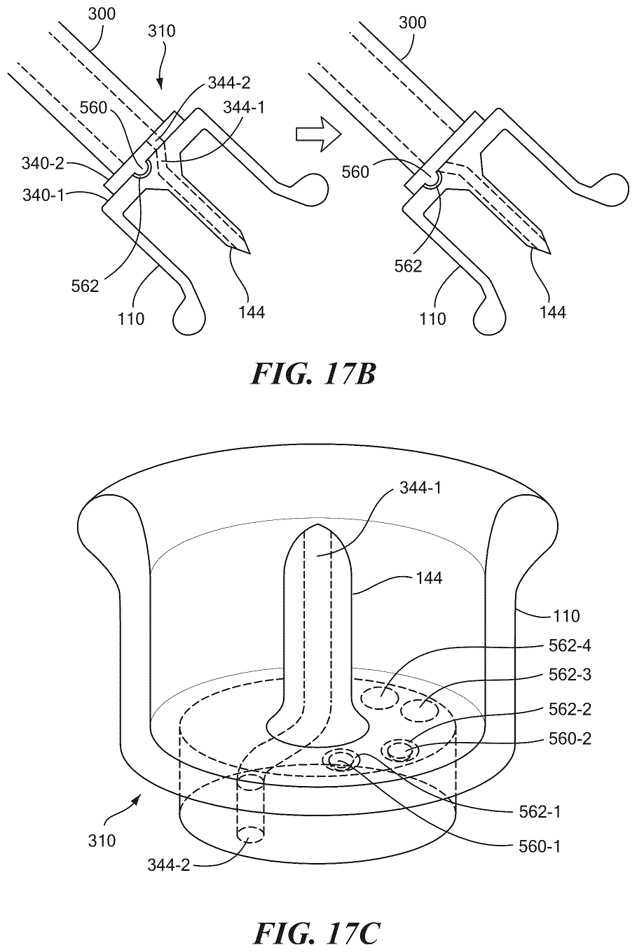

FIGS. 17A-17C show an alternate configuration having a recession and protrusion engagement on the opposed concentric sections 340 for defining valve open/closed positions. Referring to FIGS. 12-17C, the valve 310 further includes opposed surfaces on respective concentric sections 340-1 . . . 340-2 adapted for rotation in a slidable engagement. Opposed complementary surfaces on the concentric sections have one or more recessions 562 or dimples on a first opposed surface, and one or more protrusions 560 on a second opposed surface, such that each protrusion 560 is adapted for engaging one or more of the recessions 562 based on a resilient deformability upon alignment of the protrusion with a corresponding recession. In the example of FIGS. 17B-17C, as the concentric section 340-1, at a base of the receptacle 110, is rotated with the receptacle, recessions 562-1 . . . 562-4 (562 generally) slidably move as the concentric section 340-1 deforms slightly to allow the protrusions 560-1 . . . 560-2 (560 generally) to engage respective recessions 562. As the connector body 130 is generally formed from an injection molded polymer or similar material, the protrusions 560 resiliently deform for engagement and disengagement from the recessions 562 upon rotation of one of the concentric sections 342. In FIG. 17C, a cutaway view shows a transition as protrusions 560-1 . . . 560-2 engage recessions 562-1 . . . 562-2 when the valve 310 is in an open position, aligning the offset passage 344-1 with the offset passage 344-2.

The example of FIGS. 17A-17C shows a slant or bend in the offset passage 344-1. Such a bend provides an offset to bring the respective offset passages 344 in and out of alignment of channel portions 342. A bend in the offset passage 344 achieves a similar purpose as an offset from center of axis 320 discussed above, and either may be employed to ensure that relative rotation of concentric sections 340 brings the corresponding channel portions 342 in and out of alignment.

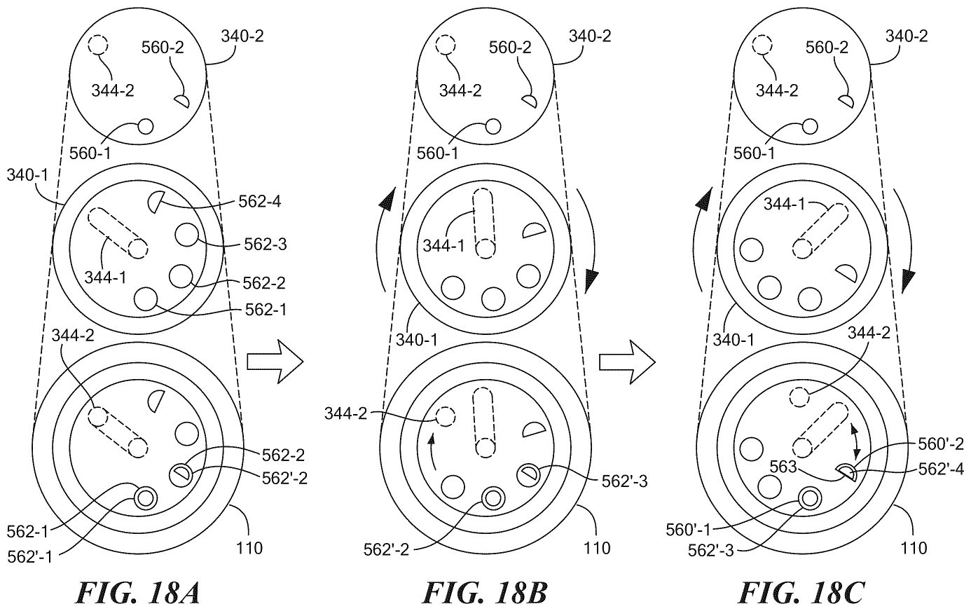

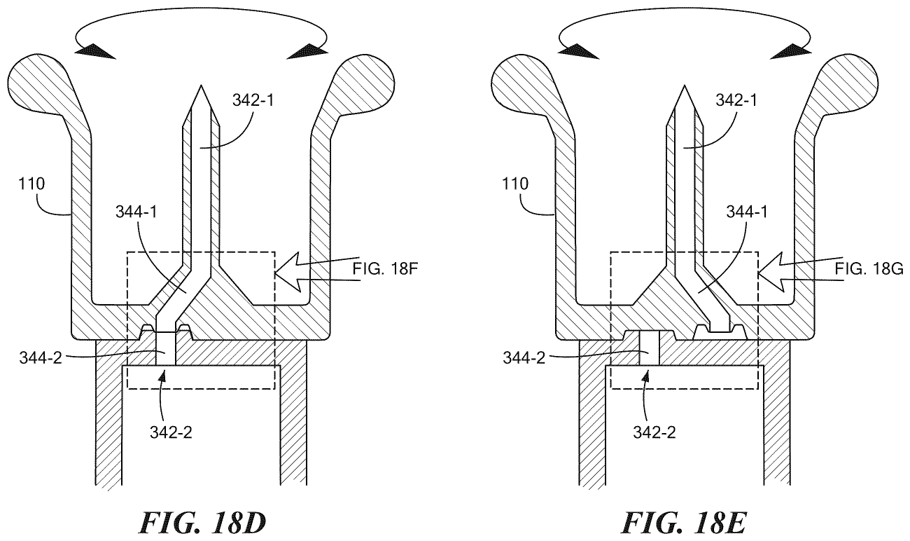

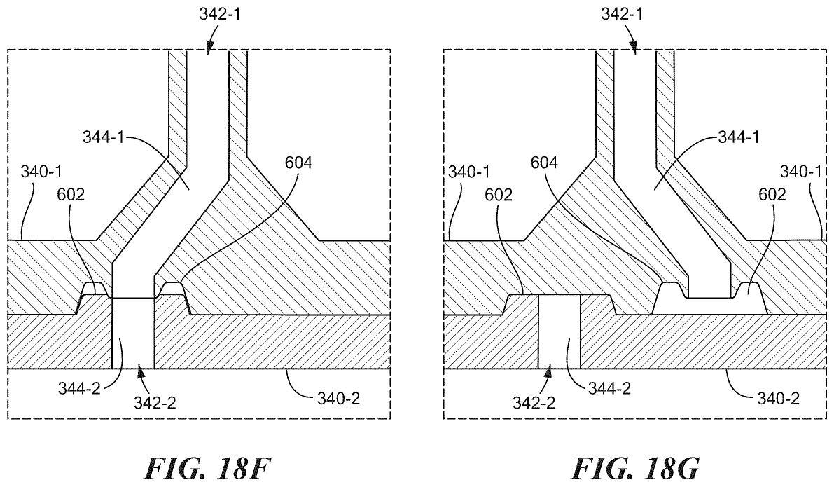

FIGS. 18A-18C show an axial view of the recessions 562 and protrusions 560 on the opposed concentric sections 342 defining the valve and locking positions. As in FIGS. 16A-16C, a locking recession defines a fixed, third valve position for enforcing single use or otherwise locking the valve closed to prevent duplicative medication administration. The locking recession 562 is adapted for a fixed engagement once engaged with the corresponding protrusion 560-2

In the configuration of FIG. 18A-18C, for each of a plurality of valve positions, a plurality of protrusions 560 (560′ when engaged) are disposed for simultaneous engagement of a corresponding plurality of recessions 562 (562′ when engaged), thereby providing a positional detent for respective positions of the valve. By engaging at least two protrusion 560 with recessions 562 corresponding to a valve position (open, closed, locked), an affirmative (“tight”) engagement of the valve is assured, without deviations or tolerance suggesting an insecure engagement.

FIGS. 18A-18C show two protrusions 560-1 . . . 560-2, and four recessions 562-1.562-4 on respective opposed concentric sections 340-1 . . . 340-2 defining the valve 310. For each of 3 valve positions of open, closed and locked, the two protrusions engage a specific set of two recessions 562. FIG. 18A shows an open position where the protrusions 560 engage recessions 562-1 and 562-2. In FIG. 18B, as the receptacle 110 is rotated to concurrent rotate concentric section 340-1, recessions 562′-2 and 562′-3 are engaged, as the offset passages 344 are brought out of alignment to close the valve 310. In a further rotation, shown in FIG. 18C, the locking position engages recessions 562-3′ and 562-4′, as the channels 344 remain unaligned and the valve 310 closed.

It can be noted that recession 562-4 is engaged only in the locked position. It can be further seen that recession 562-4 and protrusion 560-2 both have a “D” shape with a flat side 563, in contrast to their circular counterparts. Protrusion 560-2 is a locking protrusion having a flat surface adapted for forming an interference fit with the corresponding recession 562-4, thereby locking the concentric sections in a fixed orientation. Since the respective “D” shape of protrusion 560-2 and recession 562-4 align in a diametrically opposed position, once engaged, recession 562′-4 butts against locking protrusion 560′-2. Other protrusions 560 have a beveled edge, and when engaged with the locking recession 562-4, may still slidably disengage as the beveled edge rides against the flat side. Only when both flat sides of the locking protrusion 560′-2 and recession 562′-4 is the locking engagement achieved and the protrusion and recession attain a locked position and will not disengage merely from rotation of the concentric sections 342 because the protrusion 560′-2 engages in an interference fit of flat sides with recession 562′-4.

FIGS. 18D-18G show a side cutaway view of the concentric sections 340 of FIGS. 18A-18C. As shown in FIG. 18D, the first channel portion 342-1 rotates into alignment with the second channel portion 342-2 to align the offset passages 344 in the valve 310 into an open position. The concentric sections 340-1, 340-2 slidably pass in close tolerance to prevent or avoid fluid (medication) leakage. In FIGS. 18D-18G, the opposed surfaces of the concentric sections 340 include a frustoconical structure on one of the opposed surfaces adapted for engaging a mating aperture on the other of the opposed surfaces. FIGS. 18F and 18G show the enlarged sections of the concentric sections 340 showing the frustoconical structure 602 surrounding the flow portion 344-1 of the valve channel 310 and adapted to engage the mating aperture 604 in a fluidic seal for aligning the offset passages 344 for fluid communication when the valve 310 is open. A resilient seal, washer or O-ring may also be employed. The frustoconical structure 602 slidably engages and passes along the other of the opposed surface of the concentric section 340-1 in a sealing engagement when the offset passages 344 of the valve channel are not aligned, thereby sealing the valve channel 310 and contents of the vial, shown in FIG. 18G. Thus, in either an open or closed valve 310 position, liquid medication cannot leak out from the valve 310.

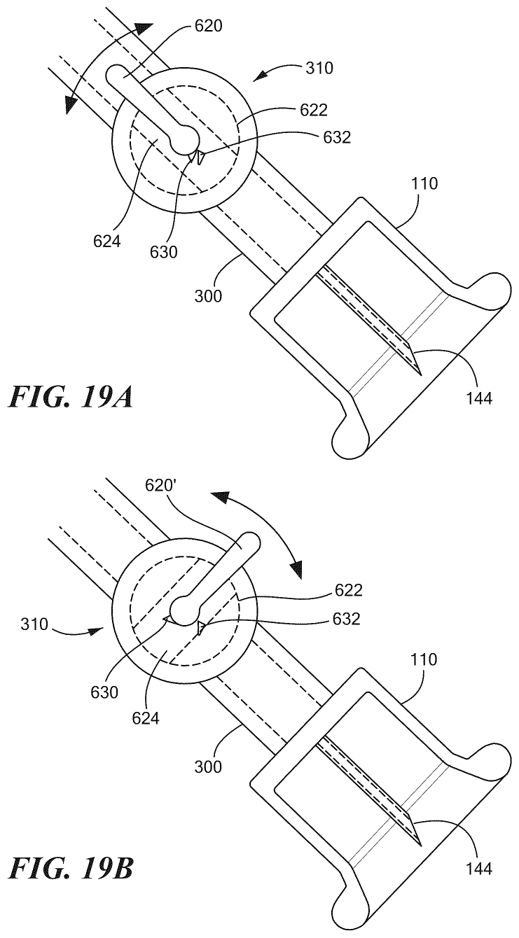

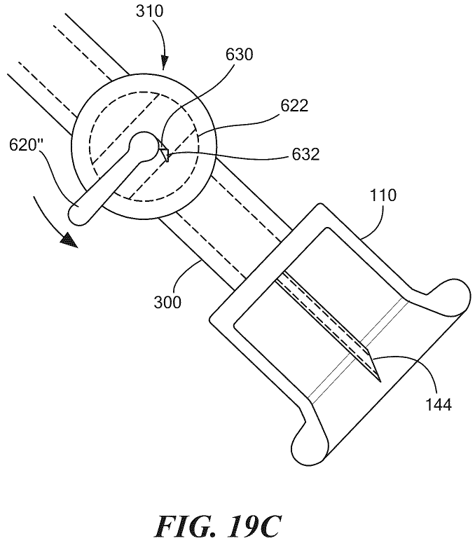

FIGS. 19A-19C show an alternate valve 310 construction in the valve channel 310. In FIGS. 19A-19C, a valving configuration provides interior valves directed from an external handle 620. The valve channel 300 includes an intermediate segment 622 having a bore 624 adapted for rotational engagement to selectively align with the valve channel 300. The intermediate segment 622 may have a spherical shape with a diametric passage defining the bore 624, where the spherical shape is rotated from the handle 620, shown as 620′ in a closed position where the bore 624 is unaligned and in an interference position with free flow though the valve channel, closing the valve and preventing medicinal flow. The handle, therefore physically turns an internal valve body in and our of alignment for opening or restricting flow.

FIGS. 19A-19C also demonstrate a locking tab 630 with a sloped or angled slide adapted to pass along a locking protrusion 632, deforming and “snapping” the valve closed as the angled surface deformably forces the locking tab 630 for passage and retracts to a closed position against a flat surface on the protrusion 632. FIG. 19B depicts a selectable valve closed position where the bore 624 is unaligned or closed, but the locking tab 630 on the handle is freely moving for allowing the handle 620′ to be moved back into open position 620. Note that the locking tab 630 is extending approximately opposite of the longitudinal dimension of the handle 620 and still travelling free away from the locking protrusion 632.

In FIG. 19C, the handle 620 is turned counterclockwise to a position 620″ opposed 180° from the selectively locked position. When the handle is turned counterclockwise to a transverse position across the flow channel 300, the locking protrusion 632 slidably engages and passes the locking tab 630 in a one-way arrangement, thereby locking the valve in a closed position. The locking tab 630 has an inkling or angled surface on the side approaching the locking protrusion 632, and a flat or butt orientation on an opposed side. As the locking tab 630 moves along the locking protrusion 632 the angled surface wedges and deforms the plastic or polymer locking protrusion 632/locking tab 630 until the angled surface slides beyond the locking protrusion 632 and “snaps” into an interference fit with the locking tab 630 for locking the valve closed.

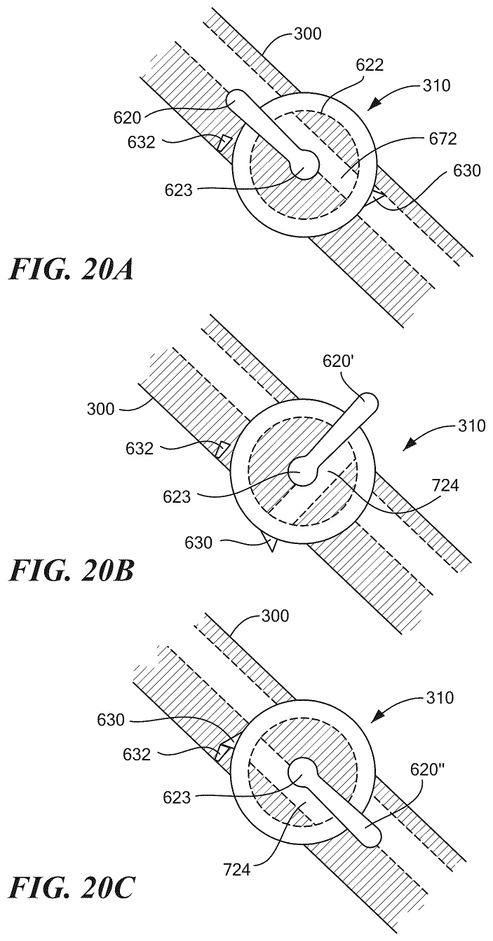

FIGS. 20A-20C show an alternate locking configuration for the valve of FIGS. 19A-19C. The intermediate segment 622 attaches to an axial shaft 623 extending to an exterior of the valve channel 300 for rotation from the handle 620. In contrast to the rotation of the receptacle 110, the axial shaft 623 rotates perpendicular to an axis through the bore 624, 724. A locking arrangement defines a third handle 620″ position locking the valve 310 in a closed position. The bore 724 is smaller than the bore 624, and in an example configuration is offset from a centerline through the valve channel 300. The valve channel 300 may also have a corresponding reduced diameter.

As the handle 620′ rotates to a closed position, the bore 724 is oriented perpendicular to an axis through the channel 300, closing the valve. However, a further handle 620″ rotation to 180° from open 620 maintains a closed position as the bore 724, aligned offset from center, does not align with the valve channel 310. However, in the locked handle 620″ position, the locking tab 630 with a sloped or angled slide passes along a locking protrusion 632, deforming and “snapping” the valve closed as the angled surface deformably forces the locking tab 630 for passage and retracts to a closed position against a flat surface on the protrusion 632. FIG. 20C shows the locked position where the locking tab has rotated clockwise beyond the locking protrusion and forms an interference fit preventing moving the handle 620″ back into either the “normal” closed 620′ or open 620 positions. An additional protrusion, lock or tab, such as on the handle, prevents defeating the locked position 620″ by merely fully rotating the handle 620 a full 360°.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A medicinal vial reconstitution and dispensing device, comprising:
a receptacle including vial holder and a spike, the spike adapted to engage a medicinal vial and adapted for penetration of a resilient portion in a cap on the medicinal vial;
an intravenous (IV) connector adapted for inline connection to a patient IV line, the IV connector adapted for transport of medicinal fluids from a fluid reservoir to a patient via the patient IV line;
a bifurcated junction body extending from the IV connector to the receptacle, the bifurcated junction body having a valve channel adapted for fluidic communication between the receptacle and the IV connector for transferring liquids between the vial and IV connector, the valve channel adapted to receive the receptacle in a rotational engagement for selective alignment with an off-center bore;
the valve channel directing fluid to the fluid reservoir before passing to the patient IV line through the IV connector at an IV output; and
a tapered end on the IV connector adapted for engaging an IV fluid bag defining the reservoir to establish fluidic communication between the IV connector and the fluid reservoir, the tapered end having first and second orifices on the tapered end, the first and second orifices defining respective fluidic connections to the patient IV line and the valve channel.

2. The device of claim 1 wherein the valve channel includes a valve for selectively opening and closing the valve channel for sealing the medicinal vial.

3. The device of claim 2 wherein the valve further comprises a plurality of concentric sections adapted to rotate individually for aligning the valve channel for fluid communication.

4. The device of claim 2 wherein the valve channel extends through a plurality of concentric sections in the body, the concentric sections defining the valve and adapted to rotate for aligning and dis-aligning the valve channel.

5. The device of claim 4 wherein the valve channel is defined by portions in each of the concentric sections, the portions offset from a rotational axis of the concentric sections for aligning the portions based on rotation of the concentric sections.

6. The device of claim 1 wherein the spike is adapted for penetration of a resilient portion in a cap on the medicinal vial when the receptacle receives the cap.

7. The device of claim 1 wherein the flow control valving element defines a plurality of fluidic flow control segments though the IV connector, the fluidic flow control segments traversed by medicinal fluid in the vial in a predetermined sequence the predetermined sequence including:
a first flow segment from a valve channel ingress to the IV connector via the valve channel,
a second flow segment from the valve channel into the fluid reservoir, and a third flow segment from the fluid reservoir to the patient IV line through the IV connector, the flow control valving element disposed for controlling fluid from the first flow segment to the second flow segment prior to the fluid traversing the third flow segment.

8. The device of claim 7 wherein the first and second flow control segments direct fluid in opposed directions though the IV connector.

9. The device of claim 7 wherein the second and third flow control segments travel though separate ingress and egress orifices between the IV connector and the reservoir.

10. The device of claim 1 wherein the IV connector has separate channels including a first channel disposed as an enclosed closed tubular volume within an enclosed tubular volume of a second channel.

11. The device of claim 1 further comprising a flange and rib formed with the IV connector at a junction of the IV connector and the valve channel, the junction formed more than half the length of the IV connector distal from a tip of the tapered end.

12. The device of claim 3 further comprising opposed surfaces on respective concentric sections adapted for rotation in a slidable engagement, further comprising:
one or more recessions on a first opposed surface; and
one or more protrusions on a second opposed surface, each protrusion adapted for engaging one or more of the recessions based on a resilient deformability upon alignment of the protrusion with a corresponding recession.

13. The device of claim 12 wherein the protrusions resiliently deform and disengage from the recessions upon rotation of one of the concentric sections.

14. The device of claim 12 further comprising a locking recession, the locking recession adapted for a fixed engagement once engaged with the corresponding protrusion.

15. The device of claim 12 further comprising, for each of a plurality of valve positions,
a plurality of protrusions disposed for simultaneous engagement of a corresponding plurality of recessions, thereby providing a positional detent for respective positions of the valve.

16. The device of claim 14 wherein the locking recession has a flat surface adapted for forming an interference fit with the corresponding protrusion, thereby locking the concentric sections in a fixed orientation.

17. The device of claim 12 further comprising a frustoconical structure on one of the opposed surfaces adapted for engaging a mating aperture on the other of the opposed surfaces, the frustoconical structure surrounding the valve channel and adapted to engage the mating aperture in a fluidic seal for aligning the valve channel for fluid communication.

18. The device of claim 17 wherein the frustoconical structure slidably engages the other of the opposed surface in a sealing engagement when the valve channel is not aligned, thereby sealing the valve channel and contents of the vial.

19. The device of claim 1 further comprising a locking tab and a protrusion, the locking tab and locking protrusion extending respectively from the receptacle and the valve channel, the locking tab and protrusion adapted for slidable engagement for locking the valve in a closed position.

20. A medicinal vial reconstitution and dispensing device, comprising:
a receptacle including a vial holder and a spike, the spike adapted to engage a medicinal vial and adapted for penetration of a resilient portion in a cap on the medicinal vial;
an intravenous (IV) connector adapted for inline connection to a patient IV line, the IV connector adapted for transport of medicinal fluids from a fluid reservoir to a patient via the patient IV line;
a bifurcated junction body extending from the IV connector to the receptacle, the bifurcated junction body having a valve channel adapted for controlling fluidic communication between the receptacle and the IV connector for transferring liquids between the vial and IV connector, the valve channel including an intermediate segment having a bore, the bore adapted for rotational engagement for selective alignment with the valve channel,
the valve channel controlling fluid to the fluid reservoir before passing to the patient IV line through the IV connector at an IV output; and
a tapered end on the IV connector adapted for engaging an IV fluid bag defining the reservoir to establish fluidic communication between the IV connector and the fluid reservoir, the tapered end having first and second orifices on the tapered end, the first and second orifices defining respective fluidic connections to the patient IV line and the valve channel.

21. The device of claim 20 wherein the intermediate segment is attached to an axial shaft extending to an exterior of the valve channel for rotation from a handle, the axial shaft rotating perpendicular to an axis through the bore for controlling flow.

22. The device of claim 1 further comprising a locking tab extending from the handle and a locking protrusion extending the valve channel, the locking tab and locking protrusion adapted for slidable engagement for locking the valve in a closed position.

23. The device of claim 22 wherein the locking tab and locking protrusion are configured for sliding, deformable unidirectional communication resulting in an interference fit.
